(19) United States
(12) Patent Application Publication
(10) Pub. No.: US 2021/0038253 A1
(43) Pub. Date: Feb. 11, 2021

(54) SPECIMEN RETRIEVAL DEVICE

(71) Applicant: Covidien LP, Mansfield, MA (US)

(72) Inventors: Jacob C. Baril, Norwalk, CT (US); Matthew A. Dinino, Newington, CT (US); George Matta, Plainville, MA (US); Kevin Desjardin, Cheshire, CT (US); Nicolette R. LaPierre, Windsor Locks, CT (US)

(21) Appl. No.: 16/905,234

(22) Filed: Jun. 18, 2020

Related U.S. Application Data
(60) Provisional application No. 62/883,166, filed on Aug. 6, 2019.

(51) Int. Cl. A61B 17/34; A61B 10/00

(52) U.S. Cl. CPC A61B 17/3417; A61B 2017/00287; A61B 10/0096

(57) ABSTRACT

The disclosure is directed to a specimen retrieval device or a kit including a specimen retrieval device for use in removing tissue from the body of a patient, in aspects, as part of a minimally invasive surgical procedure. The specimen retrieval device includes a specimen bag for placement of a tissue specimen therein. The specimen retrieval device also includes a rigid inner shaft and a linked arm at the mouth of the specimen bag. In use, the linked arm is deployed to an actuated position to open the mouth of the specimen bag.

### CROSS-REFERENCE TO RELATED APPLICATION

This application claims the benefit of and priority to U.S. Provisional Patent Application No. 62/883,166 filed Aug. 6, 2019, the entire disclosure of which is incorporated by reference herein.

### TECHNICAL FIELD

The disclosure relates generally to surgical apparatuses for use in minimally invasive surgical procedures such as endoscopic and/or laparoscopic procedures. More particularly, the disclosure relates to a surgical apparatus including a specimen retrieval device for collecting body tissue(s) and/or body fluid(s) during these procedures.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A camera or endoscope is inserted through a trocar tube to permit the visual inspection and magnification of the body cavity. The surgeon can then perform diagnostic and/or therapeutic procedures at the surgical site with the aid of specialized instrumentation, such as forceps, cutters, applicators, and the like, which are designed to fit through additional cannulas.

When removing tumor or diseased tissue from a body cavity, it is important that the tumor or diseased tissue does not come into contact with healthy or uninvolved tissue. If tissue parts have to be removed, they may be introduced into a “specimen retrieval bag” or “specimen bag” at the site where the tumor or diseased tissue has been detached from the surrounding tissue. The specimen bag is then withdrawn from the body, thereby minimizing contact of the diseased tissue with healthy tissue.

### SUMMARY

The disclosure is directed to specimen retrieval devices for use in minimally invasive surgery. In aspects, a specimen retrieval device includes a tubular body defining a longitudinal bore, the tubular body having a proximal portion, a distal portion, and a cut-out portion. The specimen retrieval device also includes a rigid inner shaft having a proximal portion, a distal portion, and a handle supported on the proximal portion of the rigid inner shaft. A drive rod having a proximal portion, a distal portion, and a handle supported on the proximal portion of the drive rod is also provided. A linked arm is connected to a distal portion of the inner rigid shaft and a distal portion of the drive rod, the linked arm including a plurality of linkages that are pivotably coupled to each other, and a specimen bag is supported on the distal portion of the inner rigid shaft and the linked arm, the specimen bag including a body and an opening. The inner rigid shaft is movable in relation to the tubular body from a non-actuated position in which the linked arm and the specimen bag are positioned in a non-deployed state within the longitudinal bore of the tubular body to an actuated position in which the linked arm and the specimen bag are moved to a position externally of the distal portion of the tubular body with the specimen bag in a non-deployed state. The drive rod is movable in relation to the tubular body from a non-actuated position in which the linked arm is positioned in its non-deployed state to an actuated position in which the linked arm is moved to its deployed state to move the specimen bag to its deployed state to form the opening of the specimen bag externally of the distal portion of the tubular body.

In aspects of the disclosure, the linked arm is connected to the distal portion of the inner rigid shaft by a joint, and the linked arm is connected to the distal portion of the drive rod by a joint.

In some aspects, the linked arm is formed of a plurality of linkages joined by a plurality of joints.

In aspects of the disclosure, a portion of the linked arm passes through the cut-out portion at the distal portion of the tubular body when the drive rod is moved in relation to the tubular body from the non-actuated position to the actuated position to form the opening of the specimen bag externally of the distal portion of the tubular body.

In some aspects, moving the drive rod in relation to the tubular body from a non-actuated position in which the linked arm is positioned in a non-deployed state to an actuated position forms a semi-circular opening of the specimen bag.

In other aspects, the inner rigid shaft supports teeth and the drive rod supports a ratchet mechanism which engages with the teeth on the inner rigid shaft to maintain the opening of the specimen bag. The ratchet mechanism may include a distal portion which engages the teeth on the inner rigid shaft, and a torsion spring which urges the distal portion of the ratchet mechanism into engagement with the teeth on the inner rigid shaft.

The specimen bag may be formed of a material selected from nylon, urethane, ripstop nylon, latex, or combinations thereof.

A kit of the present disclosure includes a component for introducing a specimen retrieval device into a patient and a specimen retrieval device of the present disclosure.

### BRIEF DESCRIPTION OF THE DRAWINGS

Aspects of the disclosed specimen retrieval device are described herein with reference to the drawings wherein:

FIG. 1 is a perspective view of a specimen retrieval device in accordance with an aspect of the disclosure in a deployed condition;

FIG. 2 is an exploded view of the specimen retrieval device shown in FIG. 1;

FIG. 3 is a perspective view showing deployment of the specimen retrieval device shown in FIG. 1;

FIG. 4 is an enlarged view of the indicated area of detail shown in FIG. 3; and

FIG. 5 is a side cross-sectional view of the specimen retrieval device of FIG. 1, depicting deployment and opening of a specimen bag.

### DETAILED DESCRIPTION

The disclosure provides a specimen retrieval device for use in minimally invasive surgical procedures. As used herein with reference to the disclosure, minimally invasive surgical procedures encompass laparoscopic procedures and endoscopic procedures, and refer to procedures utilizing trocar tubes, cannula devices, or similar devices having relatively narrow operating portions capable of insertion through a small incision in the skin.

Aspects of the disclosure may be modified for use with various methods for retrieving tissue specimens during minimally invasive surgical procedures, sometimes referred to herein as minimally invasive procedures. Examples of minimally invasive procedures include, for example, cholecystectomies, appendectomies, nephrectomies, colectomies, splenectomies, and the like.

As used herein, the term distal refers to that portion of a specimen retrieval device which is farthest from the user, while the term proximal refers to that portion of the specimen retrieval device of the disclosure which is closest to the user.

Aspects of the disclosure will now be described in detail with reference to the drawings, in which like reference numerals designate identical or corresponding elements in each of the several views. In the following description, well-known functions or constructions are not described in detail to avoid obscuring the disclosure in unnecessary detail.

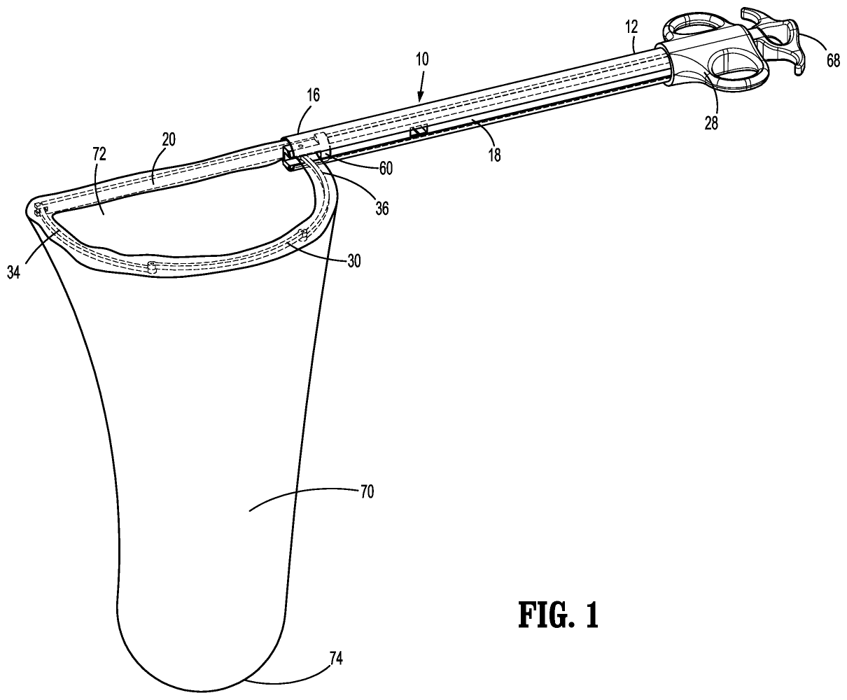

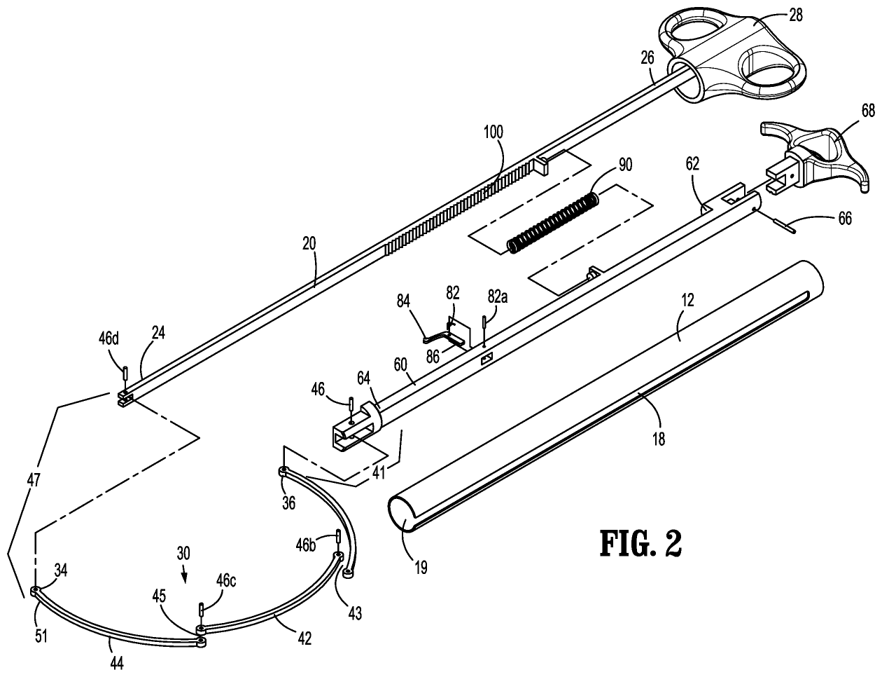

FIGS. 1-2 illustrate a specimen retrieval device 10 according to an aspect of the disclosure. The specimen retrieval device 10 includes an outer shaft 12 defining a longitudinal bore 19, a distal portion 14, a proximal portion 16, and a cut-out portion 18.

The specimen retrieval device 10 includes a rigid inner shaft 20 having a distal portion 24, a proximal portion 26, and a handle 28 at the proximal portion 26. FIG. 2 also illustrates the specimen retrieval device 10 includes a drive rod 60 having a distal portion 64, a proximal portion 62, and a handle 68 at the proximal portion 62. In aspects, the handle 68 may be affixed to the proximal portion 62 of the drive rod 60 with a pin 66.

The specimen retrieval device 10 also includes a linked arm 30 having a distal portion 34 and a proximal portion 36. The linked arm 30 is formed of linkages 40, 42 and 44 (FIG. 2), which are connected by joints 41, 43, 45 and 47. Joint 47, found at a distal portion 51 of linkage 44, connects the distal portion 34 of the linked arm 30 to the distal portion 24 of the rigid inner shaft 20. Joint 41, found at a proximal portion 53 of linkage 44, connects the proximal portion 36 of the linked arm 30 to the distal portion 64 of the drive rod 60.

Joints 41, 43, 45 and 47 may have various hinge configurations, including hinge joints (living or multi-component hinges), pivot joints, torsion spring joints, or other suitable joints. Joints 41, 43, 45 and 47 permit the linked arm 30 to deploy and open an opening or mouth 72 of the specimen bag 70 within a patient's body.

As depicted in FIG. 2, in aspects retention pins 46a, 46b, 46c, and 46d may be used to attach linkages 40, 42 and 44 to each other at joints 43 and 45, and to attach the linkage 44 to the distal portion 24 of the rigid inner shaft 20 at joint 47 and the linkage 40 to the distal portion 64 of the drive rod 60 at joint 41.

A specimen bag 70 (see, FIG. 1) is attached to the distal portion 24 of the rigid inner shaft 20 and the distal portion 34 of the linked arm 30. The specimen bag 70 may be attached to the distal portion 24 of the rigid inner shaft 20 and the distal portion 34 of the linked arm 30 by adhesive bonding, welding, formation of a cuff or cuffs, combinations thereof, and the like.

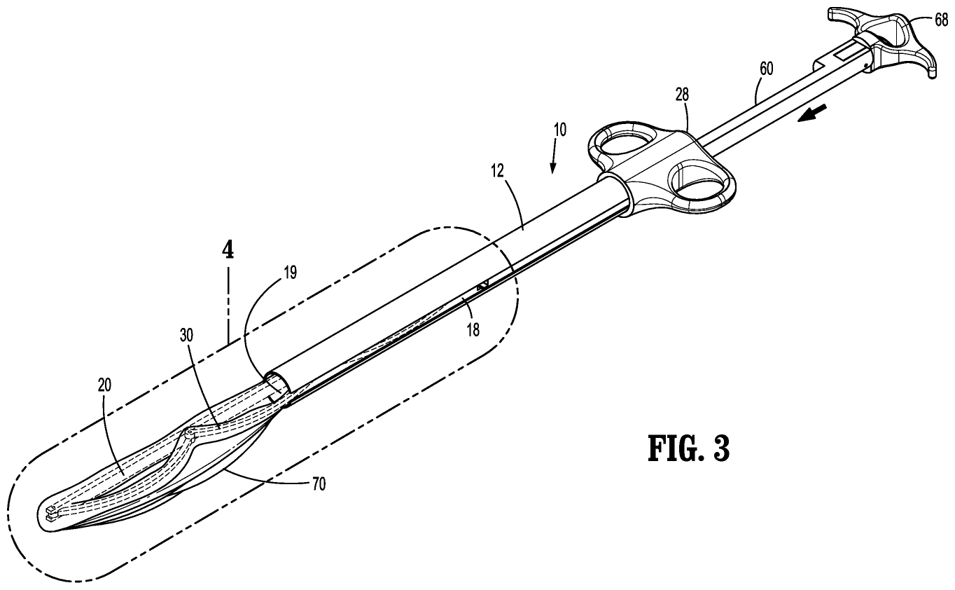

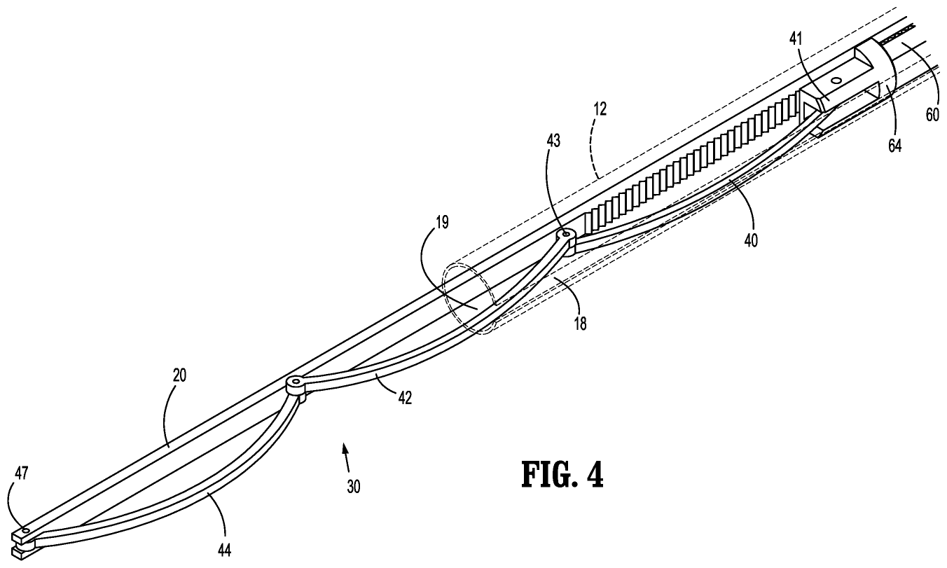

FIGS. 3-4 depict the rigid inner shaft 20, the linked arm 30, and the drive rod 60 passing within the outer shaft 12 during deployment of the specimen retrieval device 10. As shown in FIG. 3, the specimen bag 70 is compressed and/or furled for insertion into the longitudinal bore 19 of the outer shaft 12. As shown in FIG. 4, linkages 40, 42 and 44 are in a linear configuration along the same axis for insertion into the longitudinal bore 19 of the outer shaft 12. This non-actuated position of linkages 40, 42 and 44 keeps the specimen bag 70 in a closed configuration. In this manner, the rigid inner shaft 20, the linked arm 30, the specimen bag 70, and the drive rod 60 can be placed within the longitudinal bore 19 of the outer shaft 12 of the specimen retrieval device 10. The specimen retrieval device 10 is then ready to be introduced into the patient's body.

The outer shaft 12, the rigid inner shaft 20, the linked arm 30, and/or the drive rod 60 of the disclosure are made of biocompatible materials within the purview of those skilled in the art, in aspects, polymeric materials. For example, thermoplastic polyurethanes sold under the name PELLETHANE®, offer flexibility and a wide range of hardness. The outer shaft 12 and/or rigid inner shaft 20, for example, may be fabricated from PELLETHANE® 2363-80A, PELLETHANE® 2363-90A, PELLETHANE® 2363-55D, any combination thereof, or any alternatives within the purview of those skilled in the art.

The specimen bag 70 may be made from any suitable biocompatible material (e.g., nylon, urethane, ripstop nylon or latex) capable of forming a flexible collapsible member, or membrane. In aspects, the material from which the specimen bag is made is resilient, antistatic, pyrogen-free, non-toxic, and sterilizable. In aspects, materials used to form the outer shaft 12, the rigid inner shaft 20, the linked arm 30 and/or the drive rod 60 described above may be used to form the specimen bag 70. In other aspects, the specimen bag 70 is formed of materials that are different from those used to form the outer shaft 12, the rigid inner shaft 20, the linked arm 30 and/or the drive rod 60. The specimen bag 70 may be opaque, translucent, or clear.

As depicted in FIG. 1, the specimen bag 70 includes a generally tubular or elongated configuration that includes an opening 72 defined by the rigid inner shaft 20 and the linked arm 30, and a closed portion 74. Alternatively, other specimen bag configurations are envisioned.

When the specimen bag 70 is first introduced into a patient's body, the linkages 40, 42 and 44 of the linked arm 30 are disposed in a first, generally linear, orientation relative to one another, which permits their passage through the inner bore 19 of the outer shaft 12 of the specimen retrieval device 10.

As noted above, the linked arm 30 is used to form the opening 72 of the specimen bag 70. FIG. 4 depicts linkage 40 attached to the distal portion 64 of the drive rod 60. Once the rigid inner shaft 20 and the linked arm 30 have been advanced distally to introduce the specimen bag 70 into the patient's body, the drive rod 60 is then advanced distally so that linkages 40, 42 and 44 rotate about joints 41, 43, 45 and 47 thereby forming a semi-circular linked arm 30 which forms the opening 72 of the specimen bag 70. As shown in FIG. 1, the cut-out portion 18 at the distal portion 14 of the outer shaft 12 permits the deployment of linked arm 30, especially the rotation of linkage 40 at joints 41 and 43 in forming the opening 72 of the specimen bag 70.

While the linked arm 30 is depicted in FIG. 1 as a generally semi-circular shape, it is to be understood that the number of linkages and joints used to form the linked arm 30 may vary, and thus the linked arm 30 may define varying shapes, in aspects a polygonal configuration or other similar shape, to form the opening 72 of the specimen bag 70.

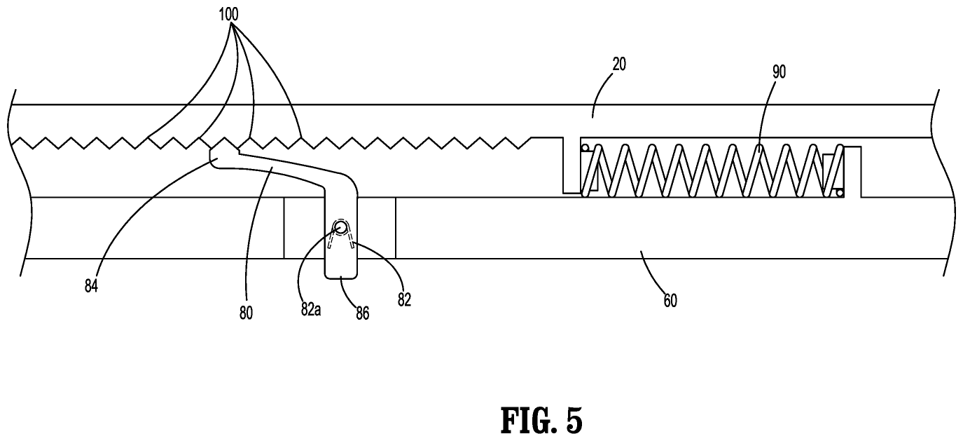

FIGS. 2 and 5 illustrate a spring 90, which keeps the linkages 40, 42 and 44 in the first, generally linear, orientation depicted in FIG. 4.

The mechanism by which the drive rod 60 is capable of deploying the linked arm 30 so that the linkages 40, 42 and 44 rotate about joints 41, 43, 45 and 47 to form the opening 72 of the specimen bag 70 is also depicted in FIGS. 2 and 5. FIGS. 2 and 5 depict the rigid inner shaft 20 having teeth 100 formed therein, with the drive rod 60 having a ratchet mechanism 80 attached thereto and a torsion spring 82 between the rigid inner shaft 20 and the ratchet mechanism 80.

As depicted in FIG. 5, when the drive rod 60 is advanced distally, the ratchet mechanism 80 affixed to the drive rod 60 engages with teeth 100 on the rigid inner shaft 20 of the specimen retrieval device 10, thereby preventing the drive rod 60 from travelling proximally, and thus forming the opening 72 of the specimen bag 70. FIGS. 2 and 5 illustrate that the ratchet mechanism 80 includes a distal portion 84 for engaging with the teeth 100 on rigid inner shaft 20 of the specimen retrieval device 10, and a torsion spring 82 which biases the distal portion 84 of the ratchet mechanism 80 to engage the teeth 100 on the rigid inner shaft 20 of the specimen retrieval device 10. As shown in FIGS. 2 and 5, a pin 82a is used to attach the torsion spring 82 to the drive rod 60.

FIG. 5 also depicts a release tab 86 at a proximal portion of the ratchet mechanism 80, which, in aspects, may be used to release the distal portion 84 of the ratchet mechanism 80 from the teeth 100 on the rigid inner shaft 20 of the specimen retrieval device 10. As shown in FIG. 2, the release tab 86 of the ratchet mechanism 80 may be accessed through the cut-out portion 18 of the outer shaft 12. After the distal portion 84 of the ratchet mechanism 80 has been released from the teeth 100 on the rigid inner shaft 20 of the specimen retrieval device 10, the spring 90 permits the return of the drive rod 60 to its retracted position and the return of the linkages 40, 42, and 44 to their linear configuration, thus closing the opening 72 of the specimen bag 10 after placement of a tissue specimen therein (not shown).

Once the specimen retrieval device of the disclosure has been removed from the patient's body, any tissue specimen may be removed from the specimen bag for further examination and the specimen bag may be discarded.

The specimen bags of the disclosure may be useful for the removal of large tissue specimens from a body cavity. The linked arm of the specimen retrieval device of the present disclosure permits the construction of larger specimen bags, having larger mouths/openings, which permit the introduction of larger tissue specimens therein.

Kits of the disclosure may include both the specimen retrieval device described above, as well as trocars, catheters, graspers, vacuum sources (tubes), combinations thereof, and the like.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto. Additionally, it is envisioned that the elements and features illustrated or described in connection with one exemplary aspect may be combined with the elements and features of another without departing from the scope of the disclosure, and that such modifications and variations are also intended to be included within the scope of the disclosure. Accordingly, the disclosure is not to be limited by what has been particularly shown and described, except as indicated by the appended claims.

What is claimed is:

1. A specimen retrieval device comprising:
a tubular body defining a longitudinal bore, the tubular body having a proximal portion, a distal portion, and a cut-out portion;
a rigid inner shaft having a proximal portion, a distal portion, and a handle supported on the proximal portion of the rigid inner shaft;
a drive rod having a proximal portion, a distal portion, and a handle supported on the proximal portion of the drive rod;
a linked arm connected to a distal portion of the inner rigid shaft and a distal portion of the drive rod, the linked arm including a plurality of linkages that are pivotably coupled to each other; and
a specimen bag supported on the distal portion of the inner rigid shaft and the linked arm, the specimen bag including a body and an opening,
wherein the inner rigid shaft is movable in relation to the tubular body from a non-actuated position in which the linked arm and the specimen bag are positioned in a non-deployed state within the longitudinal bore of the tubular body to an actuated position in which the linked arm and the specimen bag are moved to a position externally of the distal portion of the tubular body with the specimen bag in a non-deployed state, and
wherein the drive rod is movable in relation to the tubular body from a non-actuated position in which the linked arm is positioned in its non-deployed state to an actuated position in which the linked arm is moved to its deployed state to move the specimen bag to its deployed state to form the opening of the specimen bag externally of the distal portion of the tubular body.

2. The specimen retrieval device of claim 1, wherein the linked arm is connected to the distal portion of the inner rigid shaft by a joint.

3. The specimen retrieval device of claim 1, wherein the linked arm is connected to the distal portion of the drive rod by a joint.

4. The specimen retrieval device of claim 1, wherein the linked arm is formed of a plurality of linkages joined by a plurality of joints.

5. The specimen retrieval device of claim 1, wherein a portion of the linked arm passes through the cut-out portion at the distal portion of the tubular body when the drive rod is moved in relation to the tubular body from the non-actuated position to the actuated position to form the opening of the specimen bag externally of the distal portion of the tubular body.

6. The specimen retrieval device of claim 1, wherein moving the drive rod in relation to the tubular body from a non-actuated position in which the linked arm is positioned in a non-deployed state to an actuated position forms a semi-circular opening of the specimen bag.

7. The specimen retrieval device of claim 1, wherein the inner rigid shaft supports teeth and the drive rod supports a ratchet mechanism which engages with the teeth on the inner rigid shaft to maintain the opening of the specimen bag.

8. The specimen retrieval device of claim 7, wherein the ratchet mechanism includes a distal portion which engages the teeth on the inner rigid shaft, and a torsion spring which urges the distal portion of the ratchet mechanism into engagement with the teeth on the inner rigid shaft.

9. The specimen retrieval device of claim 1, wherein the specimen bag is formed of a material selected from nylon, urethane, ripstop nylon, latex, or combinations thereof.

10. A specimen retrieval device comprising:
a tubular body defining a longitudinal bore, the tubular body having a proximal portion, a distal portion, and a cut-out portion;
a rigid inner shaft having a proximal portion, a distal portion, and a handle supported on the proximal portion of the rigid inner shaft;
a drive rod having a proximal portion, a distal portion, and a handle supported on the proximal portion of the drive rod;
a linked arm connected to a distal portion of the inner rigid shaft and a distal portion of the drive rod, the linked arm including a plurality of linkages that are pivotably coupled to each other; and
a specimen bag supported on the distal portion of the inner rigid shaft and the linked arm, the specimen bag including a body and an opening,
wherein the inner rigid shaft is movable in relation to the tubular body from a non-actuated position in which the linked arm and the specimen bag are positioned in a non-deployed state within the longitudinal bore of the tubular body to an actuated position in which the linked arm and the specimen bag are deployed externally of the distal portion of the tubular body with the specimen bag in a non-deployed state, and
wherein the drive rod is movable in relation to the tubular body from a non-actuated position in which the linked arm is positioned in its non-deployed state to an actuated position in which the linked arm is moved to a deployed state to move the specimen bag to its deployed state to form a semi-circular opening of the specimen bag externally of the distal portion of the tubular body.

11. The specimen retrieval device of claim 10, wherein the linked arm is connected to the distal portion of the inner rigid shaft by a joint.

12. The specimen retrieval device of claim 10, wherein the linked arm is connected to the distal portion of the drive rod by a joint.

13. The specimen retrieval device of claim 10, wherein the linked arm is formed of a plurality of linkages joined together by a plurality of joints.

14. The specimen retrieval device of claim 10, wherein a portion of the linked arm passes through the cut-out portion at the distal portion of the tubular body when the drive rod is moved in relation to the tubular body from the non-actuated position to the actuated position to form the opening of the specimen bag externally of the distal portion of the tubular body.

15. The specimen retrieval device of claim 10, wherein the inner rigid shaft possesses teeth and the drive rod possesses a ratchet mechanism which engages with the teeth on the inner rigid shaft to maintain the opening of the specimen bag.

16. The specimen retrieval device of claim 15, wherein the ratchet mechanism possesses a distal portion which engages the teeth on the inner rigid shaft, and a torsion spring which pivots to engage the distal portion with the teeth on the inner rigid shaft.

17. The specimen retrieval device of claim 10, wherein the specimen bag is formed of a material selected from nylon, urethane, ripstop nylon, latex, or combinations thereof.

18. A kit comprising:
a component for introducing a specimen retrieval device into a patient; and
a specimen retrieval device including:
a tubular body defining a longitudinal bore, the tubular body having a proximal portion, a distal portion, and a cut-out portion;
a rigid inner shaft having a proximal portion, a distal portion, and a handle supported on the proximal portion of the rigid inner shaft;
a drive rod having a proximal portion, a distal portion, and a handle supported on the proximal portion of the drive rod;
a linked arm connected to a distal portion of the inner rigid shaft and a distal portion of the drive rod, the linked arm including a plurality of linkages that are pivotably coupled to each other; and
a specimen bag supported on the distal portion of the inner rigid shaft and the linked arm, the specimen bag including a body and an opening,
wherein the inner rigid shaft is movable in relation to the tubular body from a non-actuated position in which the linked arm and the specimen bag are positioned in a non-deployed state within the longitudinal bore of the tubular body to an actuated position in which the linked arm and the specimen bag are moved to a position externally of the distal portion of the tubular body with the specimen bag in a non-deployed state, and
wherein the drive rod is movable in relation to the tubular body from a non-actuated position in which the linked arm is positioned in its non-deployed state to an actuated position in which the linked arm is moved to its deployed state to move the specimen bag to its deployed state to form the opening of the specimen bag externally of the distal portion of the tubular body.

19. The kit of claim 18, wherein the linked arm is formed of a plurality of linkages joined by a plurality of joints, the linked arm is connected to the distal portion of the inner rigid shaft by a joint, and the linked arm is connected to the distal portion of the drive rod by a joint.

20. The kit of claim 18, wherein the inner rigid shaft possesses teeth and the drive rod possesses a ratchet mechanism which engages with the teeth on the inner rigid shaft to maintain the opening of the specimen bag.
